(19) United States
(12) Patent Application Publication
(10) Pub. No.: US 2025/0213791 A1
(43) Pub. Date: Jul. 3, 2025

(54) Flexible tubular medical device comprising near-infrared light lamp and therapeutic fluid injection needle to treat tumors and cancer cells in vivo

(71) Applicant: JIANFEI YE, MILILANI, HI (US)

(72) Inventor: JIANFEI YE, MILILANI, HI (US)

(21) Appl. No.: 18/401,613

(22) Filed: Dec. 31, 2023

(51) Int. Cl. A61M 5/178; A61M 11/06

(52) U.S. Cl. CPC A61M 5/178; A61M 11/06; A61M 2205/36; A61M 2205/50; A61M 2205/587; A61M 2205/8206

(57) ABSTRACT

A novel flexible tubular medical device for therapies for tumor in vivo or cancer cells, comprising near-infrared light lamp, a therapeutic fluid injection needle and a control panel. This invented device is to conduct a type of in vivo cancer therapy on a non-invasive or minimally invasive surgery basis. Said needle and said near-infrared light lamp are configured to be inserted into human body during a therapeutic operation, operator of this device may conduct operations of therapeutic fluid injection and near-infrared light irradiation via a control panel in vitro. Step One is to inject, or to spray, therapeutic fluid into targeted tumor in vivo, Step Two is to use the near-infrared light lamp to irradiate those targeted tumors dyed with therapeutic fluid molecules, the purpose of near-infrared light irradiation is to stimulate those therapeutic molecules attached to cancer cells membranes and cause them to vibrate in sync, those molecules originated from therapeutic fluid act like millions of molecular machines, enough to tear apart a huge number of cancer cells membranes by mechanical forces to shrink or eliminate tumors.

### CROSS-REFERENCE TO RELATED APPLICATIONS

Not Applicable

### STATEMENT REGARDING FEDERALLY SPONSORED RESEARCH OR DEVELOPMENT (IF APPLICABLE)

Not Applicable

### REFERENCE TO SEQUENCE LISTING, A TABLE, OR A Internet PROGRAM LISTING COMPACT DISK APPENDIX (IF APPLICABLE)

Not Applicable

### BACKGROUND OF THE INVENTION

Over past decades the number of tumor & cancer cases rose significantly world wide, contributing a major public health crisis of modern world. However there are very limited methods to cure cancer completely.

In recent a new fact has been discovered to destroy cancer cells, that is to use a certain category of therapeutic chemical ingredients in liquid form, mostly a variety of bio-imaging dyes, e.g. aminocyanine molecules, to dye and to attach to cancer cells, then to utilize near-infrared light to stimulate those molecules to vibrate in sync, enough to break apart the membranes of cancer cells. Aminocyanine molecules have been used in bio-imaging as synthetic dyes for many years. They stay stable in water and are very good at attaching themselves to the outside of cells.

Such a new discovery of cancer killing mechanism is based on law of physics, the structure and chemical properties of this type of therapeutic chemical ingredients. e.g. aminocyanine molecules, enable them to stay in sync with the right stimulus, such as near-infrared light. When in motion, the electrons inside the molecules form what's known as plasmon, collectively vibrating entities that drive movement across the whole of the molecule. It is similar with a situation that millions of “molecular jackhammers” vibrate in sync and tear apart every membrane of cancer cells attached to those “molecular jackhammers”.

Multiple experiments have been conducted on experimental mice and in vitro cancer cells in petri dish to validate the effectiveness of combined utilization of aminocyanine with near-infrared light on curing cancers. In an animal clinic trial involving experimental mice, 50% mice with melanoma tumors got cured completely. In another medical experiment, 99% of cancer cells in vitro in a petri dish were destroyed after being dyed by aminocyanine then being irradiated with near-infrared light. Because this method of killing cancer cells is based on mechanical vibration, it is very difficult for cancer cells to evolve blockage or tolerance against this kind of treatment.

In the past effectiveness experiments, researchers could only use near-infrared light lamp to irradiate tumor in vitro (such as melanoma cancer on skin of experimental mice) or cancer cells (such as melanoma cancer clusters in a petri dish) to test the effectiveness of this cancer-killing mechanism. Upon the moment while this patent application is being written, there is no effectiveness validation experiments conducted for tumor in vivo, also there is no research paper published to document such an effectiveness validation experiments on tumor in vivo. The reason of lacking tumor in vivo effectiveness experiments is obvious: based on the principles of physics, even if near-infrared light can penetrate and propagate about 10 centimeters within human tissue, its light intensity will still be greatly weakened. For tumors in the body, such as kidney cancer, liver cancers, stomach cancer etc., scientific researchers and the medical devices market have not been offered with such a medical device which is configured to enter the human body through non-invasive or minimally invasive incision to effectively treat tumors in vivo by said “molecules-machine” in mechanism. Both medical device market and medical research institutions desperately need such a novel medical device which is configured to enter the human body on a non-invasive or minimally invasive surgery basis, and complete above mentioned two-step procedure involving dyeing cancer cells then directly irradiate cancer cells via near-infrared light at close range. Said novel device may bring significant efficiency and ease to near-infrared light related cancer killing therapy in vivo. This kind of novel medical device, if any, will kills tumors in vivo more effectively and makes in vivo tumors shrink or even disappear.

This invention is a flexible tubular medical device comprising near-infrared light lamp and a therapeutic fluid injection needle or spray head, typically on a non-invasive or minimally invasive surgery basis to enter the human body for said cancer killing therapy.

Herein non-invasive surgery are defined as a type of surgery using medical procedures which do not involve incisions on the skin. Meanwhile minimally invasive surgery are defined as a type of surgery that uses smaller incisions and shorter recovery times.

Near-infrared light generally refers to light within wavelengths from 800-2500 nm. Near-Infrared light is the section of electromagnetic radiation wavelengths nearest the normal rage but just past what human eyes can see. Herein said near-infrared light lamp is defined as a lamp or a set of lamps that emit near-infrared light. In a most of application scenarios related with this invention, said lamp emits near-infrared light toward one direction with a focus.

### BRIEF SUMMARY OF THE INVENTION

The invented medical device is designed to be typically used on a non-invasive or minimally invasive surgery basis to conduct dyeing and near-infrared irradiation toward tumors in vivo, to kill a majority of, if not all, cancer cells of a tumor in vivo. Said invented medical device is flexible and tubular, comprising a near-infrared light lamp and a therapeutic fluid injection needle or spray head to cure tumor in vivo and cancer. Herein said therapeutic fluid injection needle is used to inject aminocyanine fluid or another substitute therapeutic fluid into tumor(s), said or spray head is used to spray aminocyanine fluid or another type of substitute therapeutic fluid onto the surface of tumor(s).

The invented medical device is in a shape of flexible tube typically made from non-toxic or low-toxic polymer composite materials, and/or metal, or another other bio-compatible materials. There are near-infrared light lamp and injection needle or spray head attached at one end of said flexible tubular device (usually it is called “in vivo end”) meanwhile at another end there are control panel, battery and a chamber containing therapeutic fluid (usually it is called “in vitro end”). Said therapeutic fluid flows from said fluid chamber to injection needle or spray head via a thin inner medical plastics pipe which is wrapped inside by said tubular medical device itself. Said inner pipe is typically made of medical-use polymer composite materials, disposable and replaceable. Said therapeutic fluid is driven to flow by a small electrical or pneumatic pump which typically comprises a small pump driven by electrical motor, or a pneumatic extrusion apparatus. Herein said battery at in vitro end supplies electrical power not only to said electrical motor, if any, but also supply electrical power to near-infrared light lamp via electrical wires wrapped inside by said flexible tubular medical device itself.

A simplified version of this invention does not include electrical or pneumatic pump, as well as said therapeutic fluid storage chamber, instead, said inner medical plastics pipe offers an inlet at in vitro end so that an operator of this device may use medical syringe to inject therapeutic fluid through this inlet then flow into tumor in vivo via said needle.

In an upgraded version of this invention, there is also a heating component e.g. a heating wire, powered by said battery, so that therapeutic fluid can be heated to certain degree to be injected into tumor in vivo, to better eliminate in vivo tumor by combining mechanical vibration and hyperthermia therapy. Also, a mini version of this invention could be used in intracranial surgery or intracranial therapy, to treat a variety of brain tumors.

During non-invasive or minimally invasive surgery, an operator of this invented flexible tubular medical device, usually a surgeon, inserts said device's in vivo end which is attached with said near-infrared light lamp and said injection needle or spray head into the human body, then continues to insert and to manipulate said flexible tube to move said near-infrared light lamp and injection needle or spray head to a proximity of an tumor in vivo to conduct related dyeing operations and subsequent near-infrared light irradiation.

### BRIEF DESCRIPTION OF THE SEVERAL VIEWS OF THE DRAWING

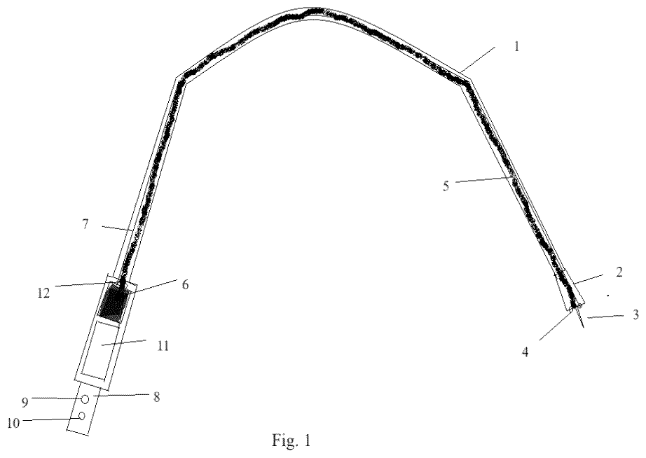

FIG. 1: This figure provides an overview of this invented flexible tubular medical device for tumor & cancer in vivo therapy. This figure also shows its major structure features and major components, highlighting the therapeutic fluid injection needle 3, near-infrared light lamp 2,4, battery 11, therapeutic fluid chamber 6, inner therapeutic fluid pipe 5, and control panel 8, 9, 10.
1—Outer Protective Pipe of Flexible Tubular Device, made from bio-compatible materials; 2—Protective External Shell of Near-infrared Light Lamp; 3—Therapeutic Fluid Injection Needle; 4—LED Bulb of Near-Infrared Light Lamp; 5—Inner Medical Plastic Pipe for Therapeutic Fluid Flow; 6—(Optional) Therapeutic Fluid Storage Chamber; 7—Electric Wire, for power supply from (optional) battery to Near-Infrared Light Lamp; 8—Main body of Control Panel; 9—Button: Therapeutic Fluid Injection on and off; 10—Button: Near-Infrared Light Lamp on and off; 11—(Optional) Battery; 12—(Optional) Electrical Pump or (Optional) Pneumatic Pump.

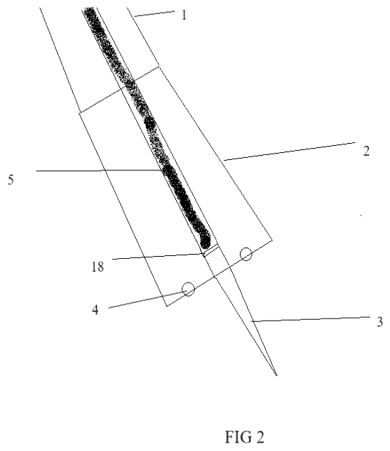

FIG. 2: This figure showcases a detailed view of said therapeutic fluid injection needle 3 and near-infrared light lamp 2,4, at in-vivo-end of this invented device, as well as a detailed structure of in-vivo-end of this invented device comprising Inter-connector 18 between the therapeutic fluid injection needle 3 and the inner medical plastics pipe 5 for controlled therapeutic fluid flow;
1—Outer Protective Pipe of Flexible Tubular Device, made from bio-compatible materials; 2—Protective External Shell of Near-infrared Light Lamp; 3—Therapeutic Fluid Injection Needle; 4—LED Bulb of Near-Infrared Light Lamp; 5—Inner Medical Plastic Pipe for Therapeutic Fluid Flow; 18—Inter-connector, between the injection needle and the inner medical plastics pipe.

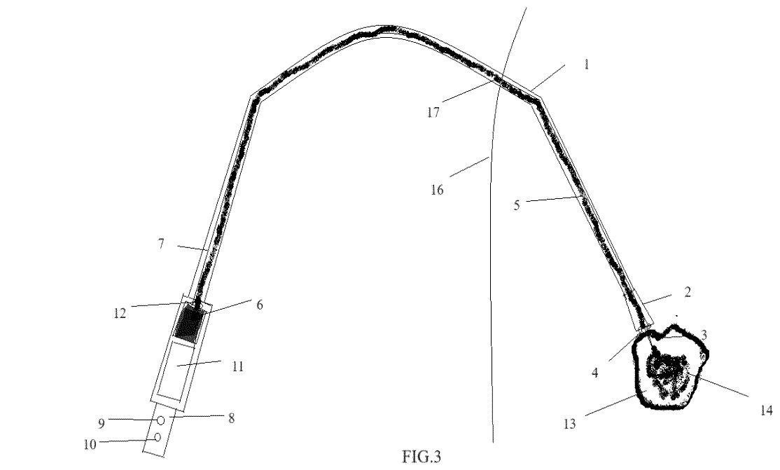

FIG. 3: This figure demonstrates a scenario that the therapeutic fluid injection needle 3 penetrates a tumor in vivo 13 then injects therapeutic fluid into said tumor 14.
1—Outer Protective Pipe of Flexible Tubular Device, made from bio-compatible materials; 2—Protective External Shell of Near-infrared Light Lamp; 3—Therapeutic Fluid Injection Needle; 4—LED Bulb of Near-Infrared Light Lamp; 5—Inner Medical Plastic Pipe for Therapeutic Fluid Flow; 6—(Optional) Therapeutic Fluid Storage Chamber; 7—Electric Wire, for power supply from (optional) battery to Near-Infrared Light Lamp; 8—Main body of Control Panel; 9—Button: Therapeutic Fluid Injection on and off; 10—Button: Near-Infrared Light Lamp on and off; 11—(Optional) Battery; 12—(Optional) Electrical Pump or (Optional) Pneumatic Pump. 13—Tumor in vivo; 14—Dyed Cancer Cells inside tumor; 16—Skin Boundary of Patient's Body; 17—a Small Incision on human skin, during a minimally-invasive surgery.

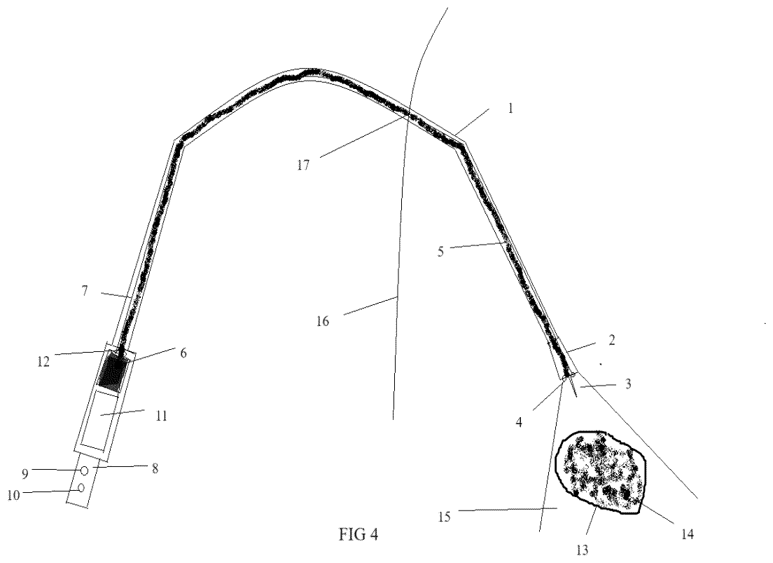

FIG. 4: This figure demonstrates a scenario that said near-infrared light lamp 2,4 is irradiating a tumor in vivo 13, a moment after therapeutic fluid being injected into said tumor 14.
1—Outer Protective Pipe of Flexible Tubular Device, made from bio-compatible materials; 2—Protective External Shell of Near-infrared Light Lamp; 3—Therapeutic Fluid Injection Needle; 4—LED Bulb of Near-Infrared Light Lamp; 5—Inner Medical Plastic Pipe for Therapeutic Fluid Flow; 6—(Optional) Therapeutic Fluid Storage Chamber; 7—Electric Wire, for power supply from (optional) battery to Near-Infrared Light Lamp; 8—Main body of Control Panel; 9—Button: Therapeutic Fluid Injection on and off; 10—Button: Near-Infrared Light Lamp on and off; 11—(Optional) Battery; 12—(Optional) Electrical Pump or (Optional) Pneumatic Pump. 13—Tumor in vivo; 14—Dyed Cancer Cells inside tumor; 15—Light Cone of Near-Infrared Light Irradiation; 16—Skin Boundary of Patient's Body; 17—a Small Incision on human skin, during a minimally-invasive surgery.

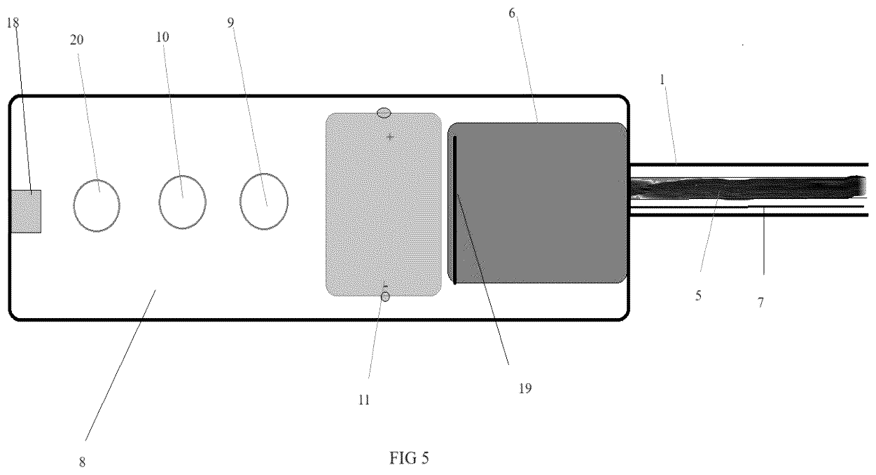

FIG. 5: This figure displays a control panel 8's interface & structure, showing major control buttons 9,10, 20 available to an operator, as well as (optional) battery 11, external electric power inlet 18, (optional) Therapeutic Fluid Storage Chamber, (Optional) Heating Component which is an electric heating wire 19.
1—Outer Protective Pipe of Flexible Tubular Device, made from bio-compatible materials; 5—Inner Medical Plastic Pipe for Therapeutic Fluid Flow; 6—(Optional) Therapeutic Fluid Storage Chamber; 7—Electric Wire, for power supply from (optional) battery to Near-Infrared Light Lamp; 8—Main body of Control Panel; 9—Button: Therapeutic Fluid Injection on and off; 10—Button: Near-Infrared Light Lamp on and off; 11—(Optional) Battery; 20—(Optional) Electric Heating Button, for heating therapeutic fluid.

In conclusion, the flexible tubular device described herein provides a novel approach to in vivo tumor treatment. By combining aminocyanine or any other substitute therapeutic chemical ingredient, and near-infrared light, this device offers a non-invasive or minimally invasive solution for selectively targeting and destroying cancer cells of tumor in vivo.

### DETAILED DESCRIPTION OF THE INVENTION

This invention is a flexible tubular medical device capable of killing cancer cells of tumor(s) in vivo by using therapeutic fluid at first then applying near-infrared light irradiation second.

Said therapeutic fluid contains aminocyanine molecules, or any other substitute molecules which is a bio-compatible and good attaching to membranes of cancel cells and can be stimulated by near-infrared light to vibrate in sync on membranes, enough to break apart those membranes of cancel cells.

This invented flexible tubular device is also made from bio-compatible materials, ensuring its compatibility with the human body. The material selected also gives this flexible tubular device a certain degree of stiffness, so that an operator of this device may easily manipulate its motion in vivo by holding its in vitro portion.

This invented device consists of a flexible tubular structure 1 with a therapeutic fluid injection needle 3 or spray head, and a near-infrared light lamp 2, 4 at one end, while the control panel 8, therapeutic liquid chamber 6, battery 11 are located at the other end, at the same end there are also optional components including a small electrical pump or pneumatic pump 12, one of these two must be chosen if said device is not a simplified version.

A therapeutic fluid injection needle 3 can be replaced by other similar needles in different lengths, so that a best-performed needle can be chosen to fit the size of a tumor; or said needle also can be replaced by a spray head, thus therapeutic fluid being sprayed onto surface of tumor 13 or cancer cells clusters rather than being injected.

Also, therapeutic fluid injection needle 3 at one end of said invented device is connected to said fluid containing chamber 6 at another end, via a thin inner pipe 5 which is wrapped inside by said tubular medical device itself. This thin inner pipe 5 is also made from bio-compatible materials, such as medic plastics, ensuring its compatibility with the human body, and said thin pipe 5 is disposable and replaceable to ensure medical safety between two application shifts of different patients.

When this invented device is operated by a medical personnel, typically by a surgeon, its needle end, or to be called “in vivo end”, will be inserted into human body through a small incision 17 on skin of human body 16, then said medical personnel may continue to insert said invented device, which is in flexible tubular shape as previous described, further extend said device into human body, so that said near-infrared light lamp 2, 4 and injection needle 3 can be placed at a proximity of a targeted in vivo tumor 13 to conduct related dyeing operations and subsequent near-infrared light irradiation 15.

Once said injection needle 3 is operated by medical personnel to penetrate a tumor in vivo 13, said medical personnel may press Injection Button 10 on said control panel 8, so that therapeutic fluid 5 flows from said chamber to said injection needle 3 then into the tumor 13. Because said therapeutic fluid e.g. aminocyanine molecules are very good at attaching themselves to membranes of cancel cells, they are diffusive and permeable to attach membranes of all cancer cells 14 in the tumor in a short period of time, if enough dose is given.

As the next step, a short period of time after completion of injection procedure, said medical personnel may turn on near-infrared lamp button 9 on said control panel, so that those dyed cancer cells 14 will be irradiated in near-infrared light 15. With right intense of near-infrared light 15, all molecules of therapeutic fluid e.g. aminocyanine molecules start to vibrate in sync and break apart those membranes of cancer cells. This type of therapeutic treatment toward tumor(s) or cancer cells is very much based on mechanical vibration, hence it is quite impossible for cancer cells to evolve a blockage or tolerance against such a therapy.

In an upgraded version of this invented device, there is also a heating component 19 in fluid storage chamber, powered by said battery 11 or external power inlet 18, so that therapeutic fluid can be heated to certain temperature, e.g. 110 Fahrenheit degree, then be injected into in vivo tumor 13, to better eliminate tumor in vivo by combining mechanical vibration on cancer cells membranes and hyperthermia therapy, to gain a better yield on cancer-killing. Said heating component 19, said fluid storage chamber 6, said battery 11 and said control panel 8 are all at in-vitro-end of this invented device. If there is an electrical inlet 18 for external power, said electrical inlet 18 is also at in-vitro-end as well.

In an upgraded version of this invented device, there is also a camera along with said needle 3 and said near-infrared light lamp 2,4, at in-vivo-end of this invented device. Said camera, said needle 3 and said near-infrared light lamp 2,4 enter human body 16 via a small incision 17 all together at same time during a cancer killing operation, said camera transfers real-time video and/or image to a screen in vitro via wireless communication or via electrical wires, so that an operator of this invented device can observe real-time in vivo image and/or video at his/her in vitro end, to better assist his/her operations and to better determine what are next steps.

As a simplified version of this invention, battery 11 is optional so that near-infrared light lamp 2,4 relies on external electric supply via electric power inlet 18; also electrical or pneumatic pump 12 is optional, therapeutic fluid storage chamber 6 is optional as well because by this arrangement the therapeutic fluid will be no more stored in this device, said therapeutic fluid will be injected from outside by a medical syringe. The operator of this invented device is configured to use medical syringe to directly inject therapeutic fluid via an open inlet of inner medical plastic pipe 5 at in-vitro end, then therapeutic fluid driven by medical syringe to flow to needle-end then into a tumor in vivo. As a simplified version of this invention there is no need to have therapeutic fluid storage chamber and pumps of any type.

Also, there could be a mini version of this invention for application scenario of intracranial surgery or intracranial therapy, to treat a variety of brain tumors. In this application scenario, a mini version of said invented flexible tubular medical device, still very much the same structural design as normal size version, but its diameter could be as thin as 1-3 mm, may move through tiny gaps among brain tissues.

In another design, said optional heating component 19 can be outside of fluid chamber 6. In another design, said control panel 8, said battery 11, said fluid chamber 6, said optional electrical pump or pneumatic pump 12, and said optional heating component 19 may not be integrated all together as one handle 8 in vitro, but being separated in different positions along this invented flexible tubular device 1. However, all components mentioned in this paragraph should remain in vitro during said killing-cancer operation, meanwhile said injection needle 3 or spray head, said near-infrared light lamp 2,4 and said optional camera should be inserted into human body during said killing-cancer operation. There is said thin inner medical plastics pipe 5 connecting said fluid chamber with injection needle 3 or spray head for said therapeutic fluid in said pipe 5 to flow from in-vitro-end to in-vivo-end. Also there is electric wires 7 connecting said battery 11 with said near-infrared light lamp 2,4 and said optional camera for electric power supply.

Said invented device may not only be applied on human patients but also may be used for application scenario of veterinary clinic, to cure cancers in vivo for animals and pets.

1. A flexible tubular device for therapy of tumor or cancer cells in vivo, comprising a therapeutic fluid injection needle, a near-infrared light lamp, and a control panel.

2. A device of claim 1, wherein the therapeutic fluid injection needle is connected to a thin medical plastic pipe for controlled therapeutic fluid flow.

3. A device of claim 1, wherein the therapeutic fluid injection needle can be replaced by a spray head, to spray therapeutic fluid onto tumor or cancer cells in vivo.

4. A device of claim 1, wherein the near-infrared light lamp is accompanied by a set of other lamps so that they are not only configured to emit near-infrared light, but also configured to emit visible light or infrared light, to stimulate the therapeutic fluid molecules attached to cancer cell membranes to vibrate in sync.

5. A device of claim 1, wherein the control panel allows for the adjustment of light intensity and fluid flow rate.

6. A device of claim 1, further comprising a battery for powering the device.

7. A device of claim 1, further comprising a heating component to heat therapeutic fluid for an additional purpose of cancer hyperthermia therapy; and/or further comprising a camera for taking photos and videos of tumor in vivo; and/or further comprising an electrical or pneumatic pump to drive therapeutic fluid to flow at a controlled speed.
